(19) United States
(12) Patent Application Publication
(10) Pub. No.: US 2019/0046727 A1
(43) Pub. Date: Feb. 14, 2019

(54) METHOD FOR IMPROVING THE ACCURACY OF THE QUANTITY OF MEDICATION INJECTED USING AN INJECTOR PEN AND INJECTOR PEN

(71) Applicant: Biocorp Production, Issoire (FR)

(72) Inventor: Daniel Aneas, Menetrol (FR)

(21) Appl. No.: 15/673,808

(22) Filed: Aug. 10, 2017

(51) Int. Cl. A61M 5/20; A61M 5/168; A61M 5/145; A61M 5/24

(52) U.S. Cl. CPC A61M 5/20; A61M 5/168; A61M 2005/3152; A61M 5/24; A61M 5/1452

(57) ABSTRACT

A method for improving the accuracy of the quantity of medication injected using an injector pen, the injector pen comprising a needle, a cartridge provided with a piston and a motorized assembly for driving the piston in translation inside the cartridge, the motorized assembly comprising a pusher, the method comprising successive steps consisting of a) selecting a desired quantity of medication to be injected, b) inserting the needle into the patient body, c) moving the pusher in a forward direction in order to push the piston over a certain stroke, which is determined as a function of the desired quantity of medication selected at step a), and d) moving the pusher in a backward direction, so as to allow the piston to deform towards the pusher and take its original shape.

### FIELD OF THE INVENTION

The invention relates to a method for improving the accuracy of the quantity of medication injected using an injector pen. The invention also relates to an injector pen with which the method can be implemented.

### BACKGROUND OF THE INVENTION

In the medical field, some patients, such as the patients with diabetes, use an injector pen to perform their injections. US Publication No. 2015/0051538 A1 discloses an example of an injector pen.

An injector pen typically includes a removable cartridge, provided with a piston, a needle assembly and a motorized assembly for driving the piston in translation inside the cartridge, the motorized assembly comprising a pusher, capable of pushing the piston inside the cartridge.

Diabetic patients usually perform many injections a day. In order to avoid replacing or refilling the cartridge at each injection, the capacity of the cartridge is often sized to allow a plurality of injections.

Before each injection, one calculates the desired quantity of medication to be injected, in particular the desired quantity of insulin, depending on measured blood glucose. The desired quantity of medication is then inferior to the quantity of medication contained inside the cartridge.

Cartridges with concentrated insulin have been developed over the years. The advantage of such cartridges is that they enable performing a larger number of injections. Accordingly, the cartridge is replaced less frequently, meaning that the user can use its injector pen for a longer time period. However, the injected doses are much smaller than that injected with traditional formulation (non-concentrated). It is then important to precisely control the quantity of medication that is injected in the body of the patient. However, the level of precision that is obtained with the injector pens of the market is not satisfying. In particular, one observes a dripping effect at the end of the injection: one or more drops of medication keep leaving the needle and penetrate into the patient body after the injection. Accordingly, the injected dose is often superior to the desired quantity. This arises from the fact that the piston is generally made of rubber, and therefore deforms during the injection, because of friction with the wall of the cartridge. At the end of the injection, the piston takes its original shape and expels, while deforming, one or more drops of medication outside the cartridge. The associated problem is that, in the particular case of a concentrated dose, one drop of medication represents a high percentage of the total quantity of the injected dose and leads to an overdose. Such overdose is often at the origin of health issues. The same problem applies when dealing with other medication, where injected doses are very small, such as the ones used in pediatrics.

### SUMMARY

The invention is particularly intended to remedy these drawbacks by proposing a new method that enables injecting a dose of medication with a higher precision.

For this purpose, the invention relates to a method for improving the accuracy of the quantity of medication injected using an injector pen, the injector pen comprising a needle, a cartridge provided with a piston and a motorized assembly for driving the piston in translation inside the cartridge, the motorized assembly comprising a pusher. The method comprises successive steps consisting of:

a) selecting a desired quantity of medication to be injected,

b) inserting the needle into the patient body,

c) moving the pusher in a forward direction in order to push the piston over a certain stroke, which is determined as a function of the desired quantity of medication selected at step a), and

d) moving the pusher in a backward direction, so as to allow the piston to deform towards the pusher and take its original shape.

By means of the invention, by allowing the deformation of the piston towards the pusher, there is no risk of generating an over pressure in the liquid contained in the cartridge body, on the side of the medication. Accordingly, there is no risk of expelling, after the injection is complete, one or more drops of medication outside the cartridge due to the deformation of the piston.

According to advantageous but non-mandatory aspects of the invention, the method may comprise one or a plurality of the following features, taken in any permissible combination:
Step d) is implemented immediately after step c) is complete.
Step d) is implemented after a predetermined period has elapsed since step c) is complete.
Said predetermined period is between 5 s and 10 s.
The method further comprises a step consisting of sending a signal after said predetermined period has elapsed.
The signal is a visual signal or an audible signal.
The method further comprises a step consisting of sending a signal immediately after step c) is complete.
The signal is a visual signal or an audible signal.
The method comprises a step of priming before step c), said step of priming consisting of holding the injector pen vertically and in driving the piston over a short stroke to chase the air bubbles contained in the cartridge.
The injector pen is provided with at least one sensor for checking whether the injector pen is held vertically during the step of priming and/or with at least one sensor for detecting when the needle is clogged.
The method comprises a further step consisting of sending a signal and/or in inhibiting the motorized assembly of the injector pen when it is detected that the injector pen is not held vertically during the step of priming and/or when it is detected that the needle is clogged.
The injector pen is provided with at least one gyroscope for checking whether the injector pen is held vertically during the step of priming.
The injector pen is provided with an electronic control unit for detecting a rise of a motor current that typically occurs when the needle is clogged.
The cartridge includes a concentrated dose of medication.
The cartridge is removable.
The injector pen includes a human-machine interface system that enables a user to perform step a).
The human-machine interface includes a button for triggering step c).
The certain stroke is calculated by a central processing unit that is part of the injector pen.
Said desired quantity of medication is inferior to the quantity of medication contained inside the cartridge.

The invention finally relates to an injector pen, comprising a needle, a cartridge provided with a piston, a motorized assembly for driving the piston in translation inside the cartridge, the motorized assembly comprising a pusher, capable of pushing the piston inside the cartridge, a human-machine interface system that enables a user to select a desired quantity of medication to be injected, and an electronic control unit for controlling the motorized assembly, the electronic control unit being programmed to command the displacement of the pusher in a forward direction, in order to push the piston over a certain stroke, said certain stroke being determined as a function of the desired quantity of medication selected by the user and then to command the displacement of the pusher in a backward direction, so as to allow the piston to deform towards the pusher and take its original shape.

### BRIEF DESCRIPTION OF THE DRAWINGS

The invention and further advantages thereof will emerge more clearly in the light of the following description of one embodiment of a method for controlling the quantity of medication injected using an injector pen according to the principle thereof, with reference to the appended figures wherein:

FIG. 1 is a side view of an injector pen with which the method of the invention can be implemented, the injector pen comprising a cartridge provided with a piston and a motorized assembly for driving the piston in translation inside the cartridge, the motorized assembly comprising a pusher;

FIG. 2 is an enlarged view of a man-human interface of the injector pen of FIG. 1;

FIG. 3 is a cut view along line III-III of FIG. 1;

FIGS. 4-7 are cut views representing the pusher and the piston in four different configurations, respectively before the injection, during the injection, when the piston has reached the stroke end and when the piston has taken its original shape after the injection.

### DETAILED DESCRIPTION

FIG. 1 represents an injector pen 2. The injector pen 2 extends longitudinally along an axis X2.

The injector pen 2 includes a pen body 4A and a removable cap 4B to be fitted on the pen body 4A. Preferably, the pen body 4A and the pen cap 4B are made of plastic material. The pen body 4A is of tubular shape and extends along axis X2. The pen cap 4B is a classical cap comparable to that one finds on the pens used for writing. In particular, the pen cap 4B includes a clip, which enables, for example, clipping the pen on a pocket of a shirt.

Two press buttons 8A and 8B are arranged on opposite sides of the injector pen 2. Typically, the two press buttons 8A and 8B are mounted in respective cut-outs of the pen body 4A. Press button 8A bears the symbol “−”, while the press button 8B bears the symbol “+”. The two press buttons 8A and 8B are used for selecting the desired quantity of medication to be injected.

Another push button 6 is arranged at one longitudinal end of the injector pen 2, typically at the end opposite to the pen cap 4B. The push button 6 is used for triggering the injection.

All of the push buttons 6, 8A and 8B are watertight. They may be rigid or flexible.

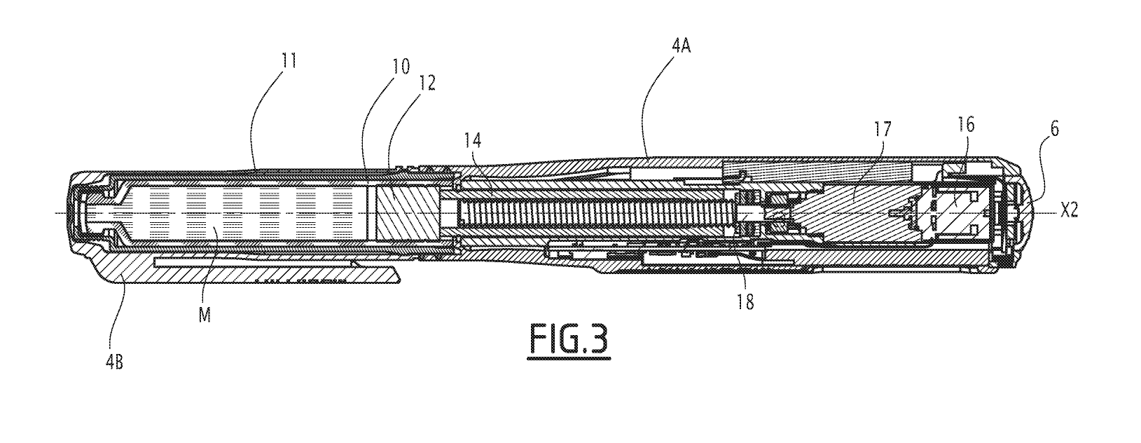

As shown on FIG. 3, the injector pen includes a cartridge 10 containing a medication M. Preferably, the medication M is in liquid form. Typically, when the pen user is diabetic, the medication is insulin and the quantity of insulin to be injected in the patient's body is determined as a function of the glucose rate in the blood. In the example, the cartridge 10 includes a concentrated dose of medication, typically a concentrated dose of insulin. In the meaning of the invention, a concentrated medication is at least twice more concentrated than a medication with a traditional formulation. In pharmacology, the international unit (IU) is a unit of measurement for the amount of a substance. The mass or volume that constitutes 1 IU varies based on which substance is being measured, and the variance is based on the biological activity or effect, for the purpose of easier comparison across substances. International units are used to quantify vitamins, hormones, vaccines, blood products, and similar biologically active substances.

For example, 1 IU of insulin is equivalent to 0.0347 mg of human insulin.

The cartridge 10 is carried in a cartridge holder 11, which is purely optional. The cartridge holder 11 is represented in FIG. 3 only. In the example, the cartridge holder 11 is removable, meaning that one shall remove the cartridge holder once the cartridge 10 is empty, in order to replace the cartridge. Typically, the cartridge holder 11 is provided with festers that enable a user to see the quantity of medication remaining in the cartridge. Further, the cartridge holder comprises, on the front side, a threaded hub for receiving a needle assembly. The needle assembly (not represented) comprises an hypodermic needle and a needle holder. Obviously, one may envisage attaching the needle assembly differently. On the rear side, the cartridge holder 11 comprises another threaded hub for attachment to the pen body 4A. Accordingly, one may easily remove the cartridge holder 11 by unscrewing the cartridge holder 11 from the pen body 4A.

In the present document, a front direction is a longitudinal direction (i.e., parallel to axis X2) oriented towards the patient body during the injection and a rear (or back) direction is the opposite direction. Typically, the needle assembly is at the front of the pen, and the push button 6 is at the back of the pen.

In practice, the needle assembly is not part of the pen as it needs to be changed at each injection.

The cartridge 10 is provided with a piston 12 that is made of elastomer, typically rubber.

Advantageously, the injector pen 2 includes a motorized assembly for driving the piston 12 in translation inside the cartridge 10. Preferably, the motorized assembly comprises a pusher 14, which is in the example a nut, and an electric motor 16, capable of driving a threaded rod 18 in rotation inside the nut 14. More precisely, the electric motor output shaft drives a gearbox 17, for example a planetary gearbox, which in turn is connected to the threaded rod 18. The nut 14 and the threaded rod 18 have complementary threads, meaning that the nut 14 moves axially when the threaded rod 18 is driven in rotation. The nut 14 is centered on the longitudinal axis X2 and the threaded rod 18 extends longitudinally.

Typically, the nut 14 may be manufactured in plastic material, while the threaded rod 18 is preferably made of steel or stainless steel.

In practice, the electric motor 16 is supplied with electricity through a battery, typically a Lithium battery, integrated to the pen. This battery may be recharged through a USB port enabling to connect the pen to a computer or to an AC outlet. Obviously, any other connecting means can be used. In practice, the battery is sized to provide an autonomy of at least one month.

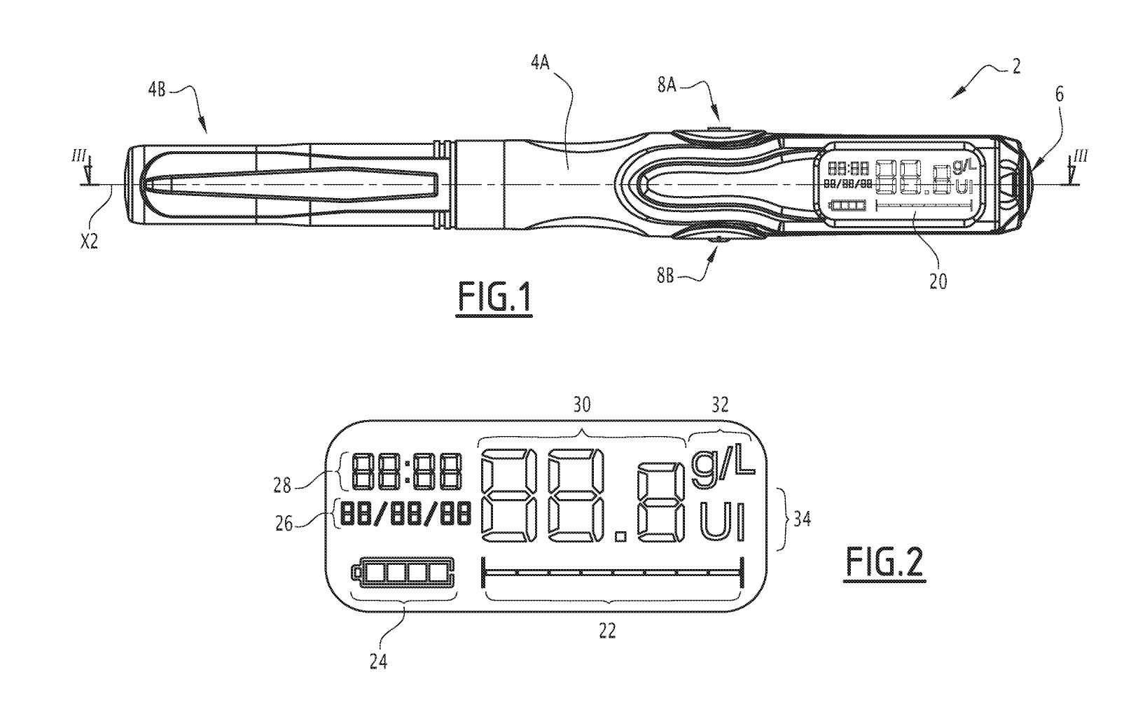

The injector pen 2 further includes a display screen 20 that is represented with a higher scale on FIG. 2. In the example, the display screen 20 is a small LCD screen. The display screen 20 is mounted in a rectangular cut-out of the pen body 4A. In practice, the display screen 20 is connected to an electronic control unit, such as a printed circuit board, or an electronic card, arranged inside the pen. Typically, the electronic control unit includes a central processing unit (CPU) or a microcontroller, an inlet port for receiving input signals, an outlet port for transmitting output signals and a memory, such as a flash memory. The electronic control unit is capable of compiling computer programs in connection with the injection process.

As shown in FIG. 2, the screen 20 includes a first bar graph 22 representative of the quantity of medication contained in the cartridge 10 and a second bar graph 24 representative of the charge rate of the injector pen battery. The display screen 12 is also configured to display the time and date (see prints 26 and 28). This display screen 12 further includes a print 30 for displaying a number to an accuracy level of a tenth. This number may be the quantity of medication to be injected or the blood glucose level. The blood glucose level is displayed in grams per liter (g/L), while the quantity of medication is displayed in an international unit (“UI” for “Unité internationale” in French).

Typically, the display screen 20 and the buttons 6, 8A and 8B form together a human-machine interface system.

Further details of the pen are described in the French Patent Application No. 3 010 908, the contents of which are hereby incorporated by reference.

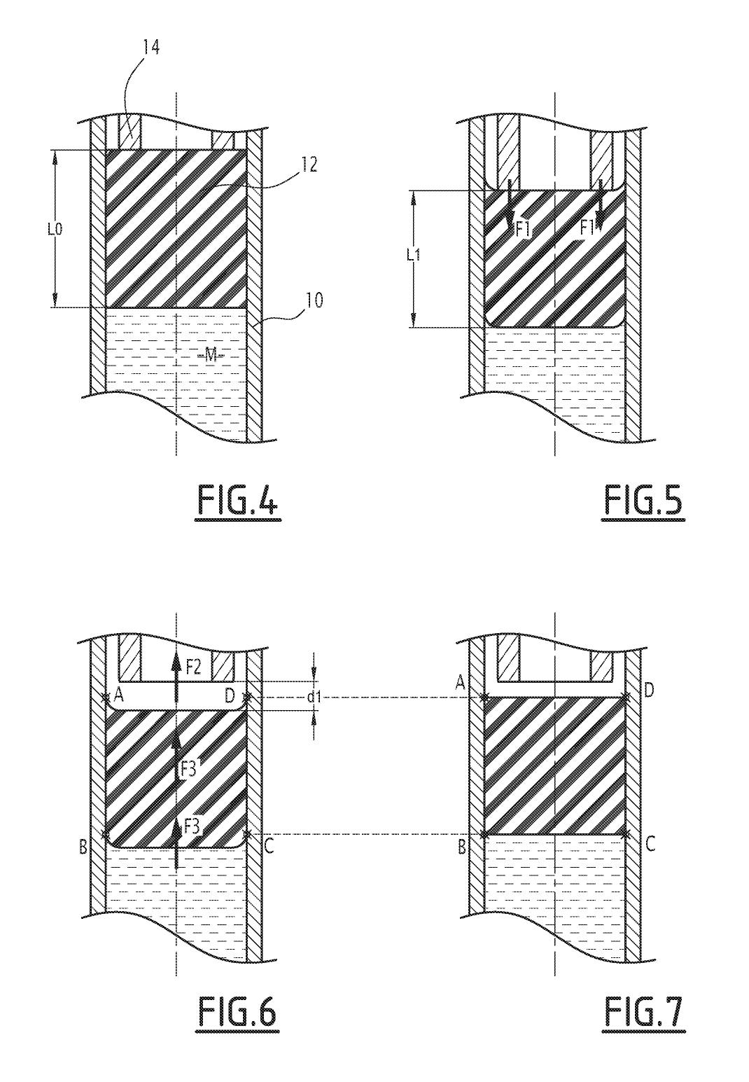

A method for controlling the quantity of medication injected using the injector pen 2 is described here-after in reference to FIGS. 4-7.

In a preliminary step, specific to the injection of insulin, the user gives notice of his blood glucose level to the electronic control unit of the pen.

Then, the patient selects, at a step a), the desired quantity of medication to be injected in his body. Typically, the patient may use alternatively the push buttons 8A and 8B to adjust the quantity of medication to be injected. By giving notice of his blood glucose level to the electronic control unit of the pen, the pen may for example check the coherence between the blood glucose level and the quantity of insulin to be injected.

The desired quantity of medication to be injected may be inferior to the quantity of medication contained inside the cartridge 10. For example, the user may select to inject 3 IU, while the cartridge contains 30 IU of medication.

In a further step, the user removes the pen cap 4B, assembles the needle assembly onto the pen and proceeds with a priming process, consisting of orienting the pen vertically, with the needle assembly at the top and in driving the piston over a short stroke to chase the air bubbles towards the exterior of the cartridge. Typically, said short stroke is identical to the stroke of the piston to inject 0.1 IU of medication. The priming process ensures that the cartridge 10 only contains liquid and that no air bubbles will be injected in the patient body. It also enables checking that the needle is not clogged.

Preferably, priming step is triggered as soon as the user depresses button 6. The priming step may be repeated by the user. In practice, the user repeats the priming step as long as no drop of medication is expelled through the needle.

Optionally, the injector pen may be provided with means for detecting that the needle is clogged. For example, such means include the central processing unit monitoring the motor current through the use of a voltmeter. Typically, the motor current may be measured by evaluating the voltage across a resistance to get the motor current. Indeed, an abnormal elevation of the motor current signifies that the motor provides an important torque, due to the fact that the needle is probably clogged. When such event occurs, the sensor may transmit a signal to the electronic control unit of the pen and an alert signal may be sent to the user for informing the user of the dysfunction. Typically, a message is displayed on the display screen 20 of the pen.

Advantageously, the pen may be provided with one or more sensors for checking whether the pen is indeed in a vertical position, with the needle pointing up, during the priming process. Typically, any orientation sensor, such as a gyroscope, may be used. In practice, an alarm signal, such as a visual signal or an audio signal is sent to the user if it is checked that the pen is not in this position during the priming process. In practice, a message is displayed on the display screen 20 of the pen to inform the user to repeat the priming step. Alternatively, the rotation of the motor can be electronically inhibited.

The user then inserts, at a step b), the needle into its body, for example in its arm or leg.

Typically, the cartridge is initially closed but is transpierced when the needle assembly is mounted onto the pen, in order to enable the liquid contained in the cartridge to flow inside the needle.

At a step c), the user depresses the button 6 at the back of the injector pen 2 to start the injection. This results in moving the pusher 14 in a forward direction, as presented by the arrows F1, in order to push the piston 64 over a certain stroke. This certain stroke is determined as a function of the desired quantity of medication selected at step a). Typically, said certain stroke is calculated by a central processing unit that is part of the injector pen 2.

As shown in FIG. 5, during the injection, the piston 12 is axially compressed, i.e. along the direction that is parallel to the longitudinal axis X2. This arises from the fact that there are friction forces between the piston 12 and the inner wall of the cartridge 10. The piston 12 then deforms and takes approximately the shape represented in FIG. 5. Typically, in this configuration, the length L1 of the piston 12, measured along the longitudinal axis X2, is inferior to the length L0 of the piston 12 measured before the injection.

At a further step d), when the piston 12 has reached the end of the stroke, i.e. at the end of step c), the pusher 14 is moved in a backward direction, as represented by the arrow F2, so as to allow the piston 12 to deform towards the pusher 14 and take its original shape, as represented by the arrows F3 on FIG. 7. In other words, the pusher 14 stops exerting any pressure onto the piston 12, meaning that the piston 12 is free to deform towards the pusher 14. Accordingly, the pressure on the front side (i.e. on the side of the medication) corresponds to the pressure of the liquid remaining in the cartridge, while the pressure on the back side (i.e. on the side of the pusher 14) corresponds approximately to the atmospheric pressure. That is why the piston deforms towards the back, without creating any overpressure in the cartridge volume containing the medication. The movement of the pusher 14 in the backward direction enables releasing the pressure in the liquid contained in the cartridge 10.

This can be evidenced by referencing four contact points A, B, C and D between the piston 12 and the wall of the cartridge 10. When the piston deforms from the configuration of FIG. 6 to the configuration of FIG. 7, the contact surface of the piston 12 with the inner wall of the cartridge remains identical.

By allowing the deformation of the piston 12 towards the pusher 14, there is no risk of generating an over pressure in the cartridge body, on the side of the medication. Accordingly, there is no risk of expelling one or more drops of medication outside the cartridge 10 due to the deformation of the piston 12.

Typically, the pusher (or nut) 14 is moved backwards over a distance dl that is superior to 0.1 mm, approximately of 0.2 mm. In practice, the distance dl shall be long enough to enable the piston to take its original shape. The distance dl is automatically compensated during the priming process of the next injection, meaning that this does not disturb the injection sequences. More generally, any axial clearance that may arise from the movement of the pusher 14 is compensated during the priming process of the next injection. Typically, the axial clearance in the screw/nut mechanism, i.e. the axial clearance that one may observe between the threads of the nut 14 and the threads of the threaded rod 18 and/or the axial clearance in the gearbox 17 are compensated when performing, at the next injection, the priming step.

In order to move the pusher 14 backwards, the threaded rod is rotated reverse, i.e. in a direction that is opposite to that during the injection sequence.

Advantageously, step d) is implemented immediately after step c) is complete. Therefore, the liquid pressure contained inside the cartridge is immediately released and there is no risk of expelling one or more drops of medication after the piston 12 has reached an end of stroke position.

Alternatively, step d) is implemented after a predetermined period has elapsed since step c) is complete. Typically, said predetermined period is between 5 s and 10 s. This means that, during said predetermined period, one or more drops of medication may be expelled due to the remaining pressure in the liquid of the cartridge. However, the dripping effect, i.e. the quantity of medication leaving the needle, is controlled and can be compensated by shortening the stroke of the piston 12. For example, the stroke of the piston 12 may be such that the delivered quantity of medication at the end of the stroke is slightly inferior to the desired quantity of medication to be injected in the patient body. For example, one may observe a difference of 0.1 IU. In practice, this difference depends on the duration of said predetermined period. In other words, the dripping effect is compensated by a slight underdose relative to the quantity selected by the user at step a).

The above-mentioned temporization often coincides with the time at which the user withdraws the needle of the injector pen from its body. Accordingly, the pusher 14 is moved back just before the needle is withdrawn. This enables preventing any aspiration of blood into the cartridge due to the slight depression occurring in the cartridge when moving the pusher 14 back.

Advantageously, an audio and/or a visual signal is sent when the injection is complete or after said predetermined period has elapsed This signal informs the user that he may withdraw the injector pen from the skin. Typically, a message may be displayed on the display screen 20 of the pen.

In a non-represented alternative embodiment, the pen injector may be used to inject any other substance than insulin, for example hormones, vitamins, vaccines, blood products, etc.

The method according to the invention is also applicable to inject non-concentrated medication. For instance, the method is also particularly suitable in pediatrics, wherein the injected doses are very small and wherein a high level of precision regarding the quantity of medication injected is required.

The features of the embodiment and alternative embodiments may be combined together to generate new embodiments of the invention.

1. A method for improving the accuracy of the quantity of medication injected using an injector pen, the injector pen comprising a needle, a cartridge provided with a piston and a motorized assembly for driving the piston in translation inside the cartridge, the motorized assembly comprising a pusher,
the method comprising successive steps consisting of:
a) selecting a desired quantity of medication to be injected;
b) inserting the needle into the patient body;
c) moving the pusher in a forward direction in order to push the piston over a certain stroke, which is determined as a function of the desired quantity of medication selected at step a); and
d) moving the pusher in a backward direction, so as to allow the piston to deform towards the pusher and take its original shape.

2. The method according to claim 1, wherein step d) is implemented immediately after step c) is complete.

3. The method according to claim 1, wherein step d) is implemented after a predetermined period has elapsed since step c) is complete.

4. The method according to claim 3, wherein said predetermined period is between 5 s and 10 s.

5. The method according to claim 3, further comprising a step consisting of sending a signal after said predetermined period has elapsed.

6. The method according to claim 5, wherein the signal is a visual signal or an audible signal.

7. The method according to claim 1, further comprising a step consisting of sending a signal immediately after step c) is complete.

8. The method according to claim 7, wherein the signal is a visual signal or an audible signal.

9. The method according to claim 1, comprising a step of priming before step c), said step of priming consisting of holding the injector pen vertically and in driving the piston over a short stroke to chase the air bubbles contained in the cartridge.

10. The method according to claim 9, wherein the injector pen is provided with at least one sensor for checking whether the injector pen is held vertically during the step of priming and/or with at least one sensor for detecting when the needle is clogged.

11. The method according to claim 10, comprising a further step consisting of sending a signal and/or in inhibiting the motorized assembly of the injector pen when it is detected that the injector pen is not held vertically during the step of priming and/or when it is detected that the needle is clogged.

12. The method according to claim 10, wherein the injector pen is provided with at least one gyroscope for checking whether the injector pen is held vertically during the step of priming.

13. The method according to claim 10, wherein the injector pen is provided with an electronic control unit for detecting a rise of a motor current that typically occurs when the needle is clogged.

14. The method according to claim 1, wherein the cartridge includes a concentrated dose of medication.

15. The method according to claim 1, wherein the cartridge is removable.

16. The method according to claim 1, wherein the injector pen includes a human-machine interface system that enables a user to perform step a).

17. The method according to claim 16, wherein the human-machine interface includes a button for triggering step c).

18. The method according to claim 1, wherein the certain stroke is calculated by a central processing unit that is part of the injector pen.

19. The method according to claim 1, wherein said desired quantity of medication is inferior to the quantity of medication contained inside the cartridge.

20. An injector pen, comprising:
a needle;
a cartridge provided with a piston;
a motorized assembly for driving the piston in translation inside the cartridge, the motorized assembly comprising a pusher, capable of pushing the piston inside the cartridge;
a human-machine interface system that enables a user to select a desired quantity of medication to be injected; and
an electronic control unit for controlling the motorized assembly, the electronic control unit being programmed to command the displacement of the pusher in a forward direction, in order to push the piston over a certain stroke, said certain stroke being determined as a function of the desired quantity of medication selected by the user and then to command the displacement of the pusher in a backward direction, so as to allow the piston to deform towards the pusher and take its original shape.
